(19) United States
(12) Patent Application Publication
(10) Pub. No.: US 2023/0045697 A1
(43) Pub. Date: Feb. 9, 2023

(54) APPARATUS FOR APPLYING A CONSUMABLE PREPARATION

(71) Applicant: VIJU BEAUTY LLC, SEDALIA, CO (US)

(72) Inventor: AVIJAH SCARBROUGH, SEDALIA, CO (US)

(21) Appl. No.: 17/548,356

(22) Filed: Dec. 10, 2021

Related U.S. Application Data
(60) Provisional application No. 63/228,871, filed on Aug. 3, 2021.

(51) Int. Cl. B05C 17/02

(52) U.S. Cl. CPC B05C 17/02

(57) ABSTRACT

The present invention provides an apparatus for applying a consumable preparation including an applicator assembly and a roller assembly. The applicator assembly includes a handle, an arm longitudinally extending from one end of the handle, and an axle connected to the arm and longitudinally spaced from the handle. The roller assembly includes a core having at least one end cap configured to rotatably receive the axle. The core extends transversely from the at least one end cap, and a solid consumable preparation is fixedly connected to at least an exterior surface of the core.

### CROSS REFERENCE TO RELATED APPLICATION

This application claims the benefit of Provisional Patent Application No. 63/228,871, filed on Aug. 3, 2021.

### BACKGROUND

This invention relates to an apparatus for applying a consumable preparation.

Consumable preparations such as sunscreen, skin moisturizing agents, makeup, deodorant, and antiperspirant, are typically sold in a variety of plastic containers. In order to apply the user must either squeeze or extend the preparation outside of the plastic container for application onto a user's skin. While plastic may make for a durable and long-lasting container, it is these same characteristics that make plastic highly resistant to decomposition and biodegradation. As a result, plastic waste has become a persistent environmental hazard, contributing to landfill and large patches of plastic floating and breaking down in the oceans.

Therefore, there is a need to solve the problems described above by providing an apparatus for applying a consumable preparation that reduces the amount of single-use plastic employed.

### BRIEF SUMMARY

The present disclosure provides an apparatus for applying a consumable preparation.

Some embodiments of the apparatus include an applicator assembly and a roller assembly. The applicator assembly includes a handle and an arm longitudinally extending from one end of the handle. An axle is connected to the arm such that it is longitudinally spaced form the handle. The roller assembly includes a core having at least one end cap configured to rotatably receive the axle. The core extends transversely from the at least one end cap, and a solid consumable preparation is fixedly connected to at least an exterior surface of the core.

According to a second aspect, the invention provides an apparatus for applying a solid consumable preparation including an applicator assembly having a handle, and at least a first arm that projects from the handle. An axle projects generally transversely from the arm longitudinally spaced form the handle. A roller assembly according to this aspect of the invention includes a generally cylindrical core configured to rotatably receive the axle and a solid consumable preparation fixedly connected to an exterior surface of the core.

Further embodiments include an apparatus for applying a consumable preparation, including an applicator assembly and a roller assembly. The applicator assembly includes a handle and an arm longitudinally extending from one end of the handle. An axle is connected to the arm such that it is longitudinally spaced form the handle. The roller assembly includes a core having at least one end cap configured to rotatably receive the axle. The core extends transversely from the at least one end cap, and a solid consumable preparation is fixedly connected to at least an exterior surface of the core. The core is generally cylindrical in shape, and the solid consumable preparation projects radially outward from the exterior surface of the core.

Further embodiments include an apparatus for applying a consumable preparation, including an applicator assembly and a roller assembly. The applicator assembly includes a handle and an arm longitudinally extending from one end of the handle. An axle is connected to the arm such that it is longitudinally spaced form the handle. The roller assembly includes a core having at least one end cap configured to rotatably receive the axle. The core extends transversely from the at least one end cap, and a solid consumable preparation is fixedly connected to at least an exterior surface of the core. The core is made from cardboard and is generally cylindrical in shape, and the solid consumable preparation projects radially outward from the exterior surface of the core.

Further embodiments include an apparatus for applying a consumable preparation, including an applicator assembly and a roller assembly. The applicator assembly includes a handle and an arm longitudinally extending from one end of the handle. An axle is connected to the arm such that it is longitudinally spaced form the handle. The roller assembly includes a core having at least one end cap configured to rotatably receive the axle. The core extends transversely from the at least one end cap, and a solid consumable preparation is fixedly connected to at least an exterior surface of the core. The core is perforated and fixed within the solid consumable preparation such that the core is disposed intermediate a radially exterior surface and a radially exterior surface of the solid consumable preparation.

Further embodiments include an apparatus for applying a consumable preparation, including an applicator assembly and a roller assembly. The applicator assembly includes a handle and an arm longitudinally extending from one end of the handle. An axle is connected to the arm such that it is longitudinally spaced form the handle. The roller assembly includes a core having at least one end cap configured to rotatably receive the axle. The core extends transversely from the at least one end cap, and a solid consumable preparation is fixedly connected to at least an exterior surface of the core. The core is made from recycled plastic and is perforated and fixed within the solid consumable preparation such that the core is disposed intermediate a radially exterior surface and a radially exterior surface of the solid consumable preparation.

Further embodiments include an apparatus for applying a consumable preparation including an applicator assembly and a roller assembly. The applicator assembly includes a handle and an arm longitudinally extending from one end of the handle. An axle is connected to the arm such that it is longitudinally spaced form the handle. The roller assembly includes a core having at least one end cap configured to rotatably receive the axle. The core extends transversely from the at least one end cap, and a solid consumable preparation is fixedly connected to at least an exterior surface of the core. The axle is fixedly connected to and projects laterally outward from the arm generally transverse to a longitudinal axis of the arm.

Further embodiments include an apparatus for applying a consumable preparation including an applicator assembly and a roller assembly. The applicator assembly includes a handle and an arm longitudinally extending from one end of the handle. An axle is connected to the arm such that it is longitudinally spaced form the handle. The roller assembly includes a core having at least one end cap configured to rotatably receive the axle. The core extends transversely from the at least one end cap, and a solid consumable preparation is fixedly connected to at least an exterior surface of the core. The solid consumable preparation projects radially outwards from the exterior surface of the core such that the solid consumable preparation has a generally cylindrical shape.

Further embodiments include an apparatus for applying a consumable preparation including an applicator assembly and a roller assembly. The applicator assembly includes a handle and an arm longitudinally extending from one end of the handle. An axle is connected to the arm such that it is longitudinally spaced form the handle. The roller assembly includes a core having at least one end cap configured to rotatably receive the axle. The core extends transversely from the at least one end cap, and a solid consumable preparation is fixedly connected to at least an exterior surface of the core. The solid consumable preparation projects outwards from the exterior surface of the core such that the solid consumable preparation has a generally spherical shape.

Further embodiments include an apparatus for applying a consumable preparation including an applicator assembly and a roller assembly. The applicator assembly includes a handle and an arm longitudinally extending from one end of the handle. An axle is connected to the arm such that it is longitudinally spaced form the handle. The roller assembly includes a core having at least one end cap configured to rotatably receive the axle. The core extends transversely from the at least one end cap, and a solid consumable preparation is fixedly connected to at least an exterior surface of the core. The solid consumable preparation has a generally elliptical shape when viewed in cross-section along a longitudinal axis of the roller assembly.

Further embodiments include an apparatus for applying a consumable preparation including an applicator assembly and a roller assembly. The applicator assembly includes a handle and an arm longitudinally extending from one end of the handle. An axle is connected to the arm such that it is longitudinally spaced form the handle. The roller assembly includes a core having at least one end cap configured to rotatably receive the axle. The core extends transversely from the at least one end cap, and a solid consumable preparation is fixedly connected to at least an exterior surface of the core. The arm is forked such that first and second portions extend laterally outwardly from a longitudinal axis of the handle, and the first and second portions extend laterally outwardly away from a longitudinal axis of the handle, and the first and second portions are spaced apart so as to receive the axle therebetween.

Further embodiments include an apparatus for applying a consumable preparation including an applicator assembly and a roller assembly. The applicator assembly includes a handle and an arm longitudinally extending from one end of the handle. An axle is connected to the arm such that it is longitudinally spaced form the handle. The roller assembly includes a core having at least one end cap configured to rotatably receive the axle. The core extends transversely from the at least one end cap, and a solid consumable preparation is fixedly connected to at least an exterior surface of the core. The arm includes one or more shafts connected so as to telescopically extend between a first length and a second length.

Further embodiments include an apparatus for applying a solid consumable preparation including an applicator assembly having a handle, and at least a first arm that projects from the handle. An axle projects generally transversely from the arm longitudinally spaced form the handle. A roller assembly according to this aspect of the invention includes a generally cylindrical core configured to rotatably receive the axle and a solid consumable preparation fixedly connected to an exterior surface of the core. The applicator assembly includes a second arm that projects longitudinally from the handle spaced laterally apart from the first arm such that the roller assembly is secured between the first and second arms.

Further embodiments include an apparatus for applying a solid consumable preparation including an applicator assembly having a handle, and at least a first arm that projects from the handle. An axle projects generally transversely from the arm longitudinally spaced form the handle. A roller assembly according to this aspect of the invention includes a generally cylindrical core configured to rotatably receive the axle and a solid consumable preparation fixedly connected to an exterior surface of the core. The applicator assembly includes a second arm that projects longitudinally from the handle spaced laterally apart from the first arm such that the roller assembly is secured between the first and second arms. The solid consumable preparation extends outwardly from the core such that the solid consumable preparation has a generally spherical shape.

Further embodiments include an apparatus for applying a solid consumable preparation including an applicator assembly having a handle, and at least a first arm that projects from the handle. An axle projects generally transversely from the arm longitudinally spaced form the handle. A roller assembly according to this aspect of the invention includes a generally cylindrical core configured to rotatably receive the axle and a solid consumable preparation fixedly connected to an exterior surface of the core. The roller assembly includes an end cap attached to one lateral end of the core which includes a bushing configured to rotatably receive the axle.

Further embodiments include an apparatus for applying a solid consumable preparation including an applicator assembly having a handle, and at least a first arm that projects from the handle. An axle projects generally transversely from the arm longitudinally spaced form the handle. A roller assembly according to this aspect of the invention includes a generally cylindrical core configured to rotatably receive the axle and a solid consumable preparation fixedly connected to an exterior surface of the core. The core is made from cardboard.

Further embodiments include an apparatus for applying a solid consumable preparation including an applicator assembly having a handle, and at least a first arm that projects from the handle. An axle projects generally transversely from the arm longitudinally spaced form the handle. A roller assembly according to this aspect of the invention includes a generally cylindrical core configured to rotatably receive the axle and a solid consumable preparation fixedly connected to an exterior surface of the core. The core is perforated and fixed within the solid consumable preparation such that the core is disposed intermediate a radially exterior surface and a radially interior surface of the solid consumable preparation.

Further embodiments include an apparatus for applying a solid consumable preparation including an applicator assembly having a handle, and at least a first arm that projects from the handle. An axle projects generally transversely from the arm longitudinally spaced form the handle. A roller assembly according to this aspect of the invention includes a generally cylindrical core configured to rotatably receive the axle and a solid consumable preparation fixedly connected to an exterior surface of the core. The axle is fixedly connected to and projects laterally outward from the first arm generally transverse to a longitudinal axis of the arm.

Further embodiments include an apparatus for applying a solid consumable preparation including an applicator assembly having a handle, and at least a first arm that projects from the handle. An axle projects generally transversely from the arm longitudinally spaced form the handle. A roller assembly according to this aspect of the invention includes a generally cylindrical core configured to rotatably receive the axle and a solid consumable preparation fixedly connected to an exterior surface of the core. The first arm includes one or more shafts configured to telescopically extend between a first length and a second length.

Further embodiments include an apparatus for applying a solid consumable preparation including an applicator assembly having a handle, and at least a first arm that projects from the handle. An axle projects generally transversely from the arm longitudinally spaced form the handle. A roller assembly according to this aspect of the invention includes a generally cylindrical core configured to rotatably receive the axle and a solid consumable preparation fixedly connected to an exterior surface of the core. The applicator includes a hook disposed on the handle longitudinally spaced from the first arm.

The apparatus for applying a consumable preparation according to the present disclosure overcomes the drawbacks of known applicators by providing a reusable applicator and a roller which contains either a recyclable, biodegradable, or reusable core to which a solid preparation is connected.

Embodiments, examples, features, aspects, and advantages of the present disclosure will become better understood with regard to the following description, appended claims and accompanying drawings.

### BRIEF DESCRIPTION OF THE DRAWINGS

The foregoing aspects and the attendant aspects of the present disclosure will become more readily appreciated by reference to the following detailed description, when taken in conjunction with the accompanying drawings, wherein:

FIG. 1 is a top view of an apparatus for applying a consumable preparation;

FIG. 2 is an exploded view of an applicator assembly and a roller assembly of the apparatus for applying a consumable preparation of FIG. 1;

FIG. 3 is a cross sectional view of the roller assembly of FIG. 2, taken along line 3-3;

FIG. 4 is a perspective view of an exemplary core of the roller assembly of FIG. 2;

FIG. 5 is a perspective view of another exemplary core of the roller assembly of FIG. 2;

FIG. 6 is a cross sectional view of the roller assembly of FIG. 5, taken along line 6-6;

FIG. 7 is a top view of another exemplary apparatus for applying a consumable preparation;

FIG. 8 is a top view of another exemplary apparatus for applying a consumable preparation; and

FIG. 9 is a top view of another exemplary apparatus for applying a consumable preparation, shown extended between a first and second length.

Reference symbols or names are used in the Figures to indicate certain components, aspects or features shown therein. Reference symbols common to more than one Figure indicate like components, aspects or features shown therein.

### DETAILED DESCRIPTION OF INVENTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

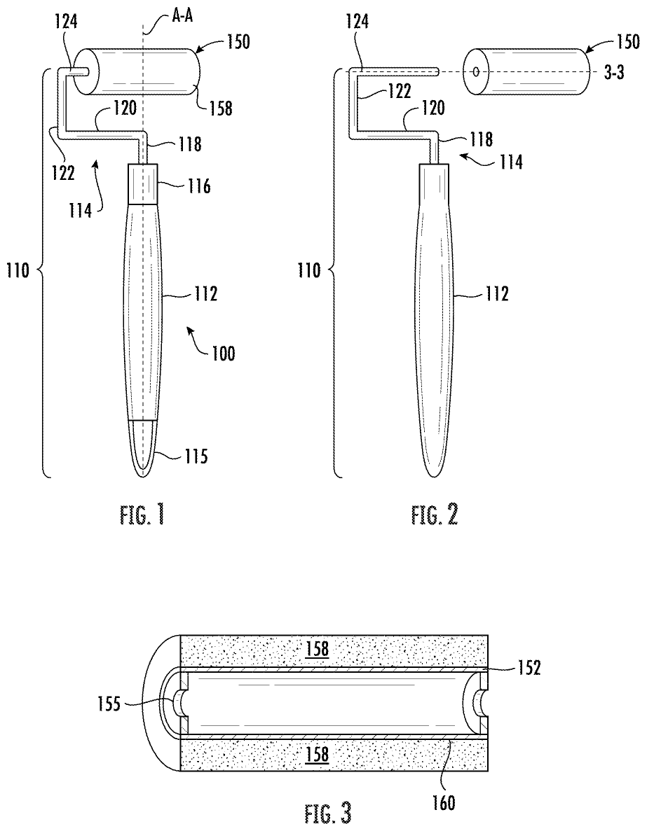

Referring to the illustrated embodiments, FIG. 1 illustrates an apparatus for applying a preparation 100 having an applicator assembly 110 and a roller assembly 150. As shown in FIG. 1, the applicator assembly 110 includes a handle 112 having a longitudinal axis A-A, and an arm 114 extends longitudinally from an end 116 of the handle 112. The handle 112 can be made from any suitable material or combination of materials in any suitable length and diameter as would be convenient and comfortable for a user to hold in a hand. As indicated, the material for the handle 112 can be stone, such as, but not limited to, jade, onyx, or quartz. In other variations, the handle can be made from cork or a tactile elastomeric material. In the embodiment illustrated in FIGS. 1 and 2, the arm 114 comprises a bent or formed rod having three approximately 90 degree bends. The arm 114 is made from metal, such as steel, but can comprise any suitable material such as wood, a dense plastic, or stone. While the arm 114 is shown as formed from a single piece, multi-piece rods are also contemplated that include multiple materials. The embodiment shown in FIG. 1 includes a hook 115 disposed on one end of the handle 112 that allows the user to hang the roller from a peg or other traditional hanging storage hardware. The hook 115 may alternative be a ring, loop, or similar apparatus that is made from either a rigid or flexible material such as metal, plastic, wood, fabric, or leather.

Referring specifically to FIG. 2, a first portion 118 of the arm 114 extends longitudinally from the handle 112 parallel and coincident with the longitudinal axis A-A of the handle 112. A second portion 120 of the arm 114 is connected to and projects generally orthogonally from an end of the first portion 118. A third portion 122 is connected to an end of the second portion and is oriented generally parallel to the longitudinal axis A-A of the handle 112 and the first portion 118. An axle 124 is connected to the arm 114 at an end of the third portion 122 such that the axle 124 is longitudinally spaced from the handle 112 and projects generally transverse to the longitudinal axis A-A of the handle 112.

The roller assembly 150 includes a core 152. In the embodiment illustrated in FIG. 3, the core 152 has an end cap 154 at a first end 156 of the core. The end cap 154 is configured to rotatably receive the axle 124 of the applicator assembly 110. The end cap 154 is connected to the core 152 at the first end 156 so that the core extends transversely from the end cap 154. The end cap 154 in the illustrated embodiment has a bushing 155 that is configured to rotatably receive the axle 124 of the assembly for smooth rotation about a longitudinal axis B-B defined by the core 152.

A solid consumable preparation 158 is fixedly connected to at least an exterior surface 160 of the core 152. The solid consumable preparation typically comprises a formulation that sluffs off as it is applied to a user's skin. The solid consumable preparation 158 may comprise sunscreen, a skin moisturizing agent, makeup, deodorant, and/or antiperspirant, although it may comprise another solid formulation as well. The solid consumable preparation may have a consistency and hardness similar to that found in stick deodorants.

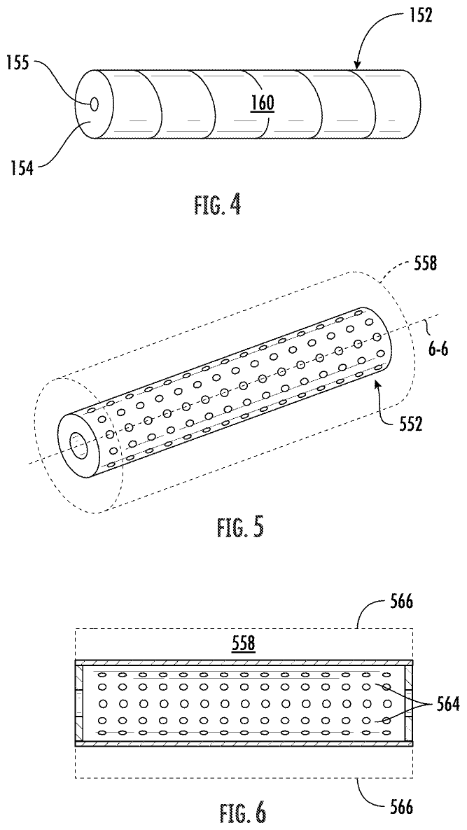

Referring to the embodiment illustrated in FIGS. 3 and 4, the core 152 is generally cylindrical in shape and comprises a cardboard tube. The consumable preparation 158 is fixedly connected to the exterior surface 160 of the core 152 (FIG. 3) and projects radially outwards from the exterior surface 160 such that the solid consumable preparation 158 has a generally cylindrical shape as best shown in FIGS. 1 and 2.

In order to apply the solid consumable preparation 158, the user holds the applicator assembly 110 by the handle 112, and applies the consumable preparation to an area of the user's body by manipulating the roller assembly 150 back and forth such that the roller assembly 150 rolls over the solid consumable preparation 158 over the area of the body, the solid consumable preparation 158 sloughs off the roller assembly 150. The solid consumable preparation 158 is rolled until the user until the solid consumable preparation 158 has sufficiently coated the area of the body to a desired degree where the more repetitions over the same area of the body will apply a greater amount of the consumable preparation 158 and vice versa.

An alternative embodiment of the core 152 is shown in FIGS. 5 and 6 where a core 552 is perforated throughout, and the core 552 is fixed within a consumable preparation 558 (shown in phantom) such that the core is disposed intermediate radially interior and radially exterior surfaces 564 and 566, respectively, of the consumable preparation 558. In this embodiment, the core 552 is made from a recycled plastic material that is capable of being recycled again after the consumable preparation 558 is entirely depleted from an exterior surface 560 of the core 552.

With reference to FIG. 8, an alternative embodiment of an assembly 800 having an applicator 810 is shown. The applicator 810 has a handle 812, and first and second arms 814 and 816, respectively. The first and second arms 814, 816 are forked such that they extend laterally outwardly and away from a longitudinal axis C-C of the handle 812 so as to form a yoke. The first and second arms 814, 816 are spaced apart from one another at ends 818, 820 such that they receive an axle 822. The ends 818, 820 define slots 824, 826 that rotatably receive the axle 822. Connectors 828 reversibly secure the axle 822 within the slots 824, 826 such that the axle 822 can be positioned in order to disconnect a roller assembly 850 and replace a core 852 with a new roller assembly having a consumable preparation 854.

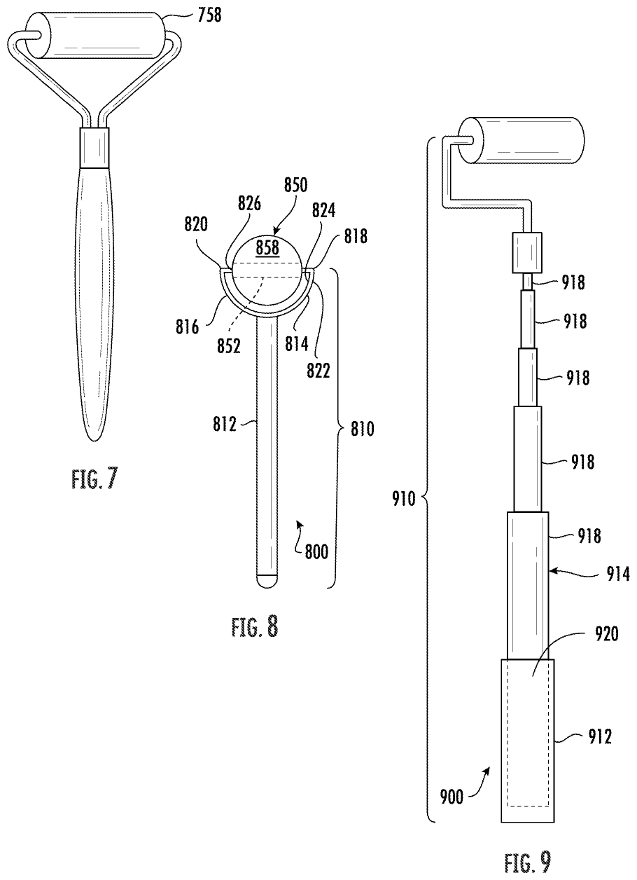

An alternative embodiment of the solid preparation is shown in FIGS. 7 and 8. A solid preparation 758 of an assembly in FIG. 7 extends from the core so as to have a generally elliptical shape in cross section. A solid preparation 858 of the embodiment in FIG. 8 extends from the core 852 so as to have a spherical shape. The shape of the solid consumable preparation may be chosen from the illustrated embodiments or those of any of a number of other configurations to suit the type of consumable preparation and/or the area of a user's body to which the consumable preparation is desirably applied. For example, a spherical or elliptical shape may be more suitable to apply deodorant or antiperspirant to a user's underarms, while a cylindrical shape may be more suitable to apply sunscreen, lotion, or makeup to a user's face.

An alternative embodiment of an apparatus 900 for applying a consumable preparation is shown in FIG. 9. The apparatus 900 includes an applicator assembly 910 that is similar in most respects to the embodiment shown in FIGS. 1 and 2. An arm 914 in the embodiment of FIG. 9 includes shafts 918 that are telescopically connected to a handle 912 and one another so that the arm 914 can extend between a first length L1 and a second length L2 (shown in phantom). The shafts 918 can be as few as a single shaft that is telescopically received within a cavity 920 defined by the handle 912 (shown in phantom). This embodiment allows a user to apply the solid consumable preparation to areas that are difficult for the user to reach such as the user's back.

Although specific embodiments of the disclosure have been described, various modifications, alterations, alternative constructions, and equivalents are also encompassed within the scope of invention as set forth in the claims.

The specification and drawings are, accordingly, to be regarded in an illustrative rather than a restrictive sense. It will, however, be evident that additions, subtractions, deletions, and other modifications and changes may be made thereunto without departing from the broader spirit and scope of invention as set forth in the claims.

1. An apparatus for applying a consumable preparation comprising:
an applicator assembly including a handle, an arm longitudinally extending from one end of the handle, and an axle fixedly connected to the arm, projecting laterally form the arm, and longitudinally spaced from the handle, so as to be supported only from the arm;
a roller assembly including a unitary core having a first end cap configured to rotatably receive the axle at a first end of the roller assembly and a second end cap disposed at a second, opposed end of the roller assembly configured to rotatably receive the axle, the axle extending through a length of the core, each end cap having an opening for receiving and directly supporting the axle therein, the core extending transversely from the at least one end cap, and a solid consumable preparation fixedly connected to at least an exterior surface of the core.

2. The apparatus of claim 1, wherein the core is generally cylindrical in shape and the solid consumable preparation projects radially outward from the exterior surface of the core.

3. The apparatus of claim 2, wherein the core is made from cardboard.

4. The apparatus of claim 1, wherein the core is perforated and fixed within the solid consumable preparation such that the core is disposed intermediate a radially exterior surface and a radially interior surface of the solid consumable preparation.

5. The apparatus of claim 4, wherein the core is made from recycled plastic.

6. (canceled)

7. The apparatus of claim 1, wherein the solid consumable preparation projects radially outwards from the exterior surface of the core such that the solid consumable preparation has a generally cylindrical shape.

8. The apparatus of claim 1, wherein the solid consumable preparation projects outwards from the exterior surface of the core such that the solid consumable preparation has a generally spherical shape.

9. The apparatus of claim 1, wherein the solid consumable preparation has a generally elliptical shape when viewed in cross section along a longitudinal axis of the roller assembly.

10. The apparatus of claim 1, wherein the arm is forked such that first and second portions are spaced apart so as to receive the axle therebetween.

11. The apparatus of claim 1, wherein the arm includes one or more shafts connected so as to telescopically extend between a first length and a second length.

12. (canceled)

13. (canceled)

14. (canceled)

15. (canceled)

16. (canceled)

17. (canceled)

18. (canceled)

19. (canceled)

20. (canceled)
